(19) United States
(12) Patent Application Publication
(10) Pub. No.: US 2019/0307121 A1
(43) Pub. Date: Oct. 10, 2019

(54) ARTICLES AND COMPOSITIONS ASSOCIATED WITH OILS ON SURFACES AND ASSOCIATED METHODS

(71) Applicant: Massachusetts Institute of Technology, Cambridge, MA (US)

(72) Inventors: Kripa K. Varanasi, Lexington, MA (US); Maher Damak, Cambridge, MA (US); Jolet De Ruiter, Copenhagen (DK)

(73) Assignee: Massachusetts Institute of Technology, Cambridge, MA (US)

(21) Appl. No.: 16/351,415

(22) Filed: Mar. 12, 2019

Related U.S. Application Data
(60) Provisional application No. 62/641,789, filed on Mar. 12, 2018.

(51) Int. Cl. A01N 25/04; A01H 5/12

(52) U.S. Cl. CPC A01N 25/04; A01H 5/12

(57) ABSTRACT

Methods related to depositing oil on surfaces and methods related to contacting surfaces at least partially covered by oil with droplets are generally provided.

### RELATED APPLICATIONS

This application claims priority under 35 U.S.C. § 119(e) to U.S. Provisional Application No. 62/641,789, filed Mar. 12, 2018, and entitled “Methods of Depositing Oil on Surfaces and Associated Articles and Systems”, which is incorporated herein by reference in its entirety and for all purposes.

### FIELD

The present invention generally relates to methods of depositing oil on surfaces, methods of contacting surfaces covered by oil with droplets, and associated articles and systems.

### BACKGROUND

Certain consumer products are designed to be applied to surfaces of interest in the form of droplets. In many cases, large fractions of the droplets applied to the surfaces bounce or roll away prior to depositing any active ingredients therein on the surfaces. This phenomenon causes consumers to apply excess amounts of the products to the surfaces, resulting in waste. Accordingly, improved methods that result in enhanced droplet retention on surfaces may be advantageous.

### SUMMARY

The present invention generally relates to compositions, articles, kits, and related methods associated with oils and surfaces, and related droplets in relation to surfaces. The subject matter of the present invention involves, in some cases, interrelated products, alternative solutions to a particular problem, and/or a plurality of different uses of one or more systems and/or articles.

Certain embodiments relate to articles. In one embodiment, an article comprises oil disposed on a surface comprising one or more protrusions. The oil is deposited from an emulsion, the emulsion comprises droplets comprising the oil dispersed within a fluid comprising water, and a ratio of an average radius of the droplets comprising the oil to an average height of the protrusions is from 0.01 to 100.

In one embodiment, an article comprises oil disposed on a surface comprising one or more protrusions. The oil is deposited from droplets comprising an emulsion. The droplets comprising the emulsion have an average radius R and an average density ρ. The emulsion comprises droplets comprising a fluid comprising the oil dispersed within a fluid comprising water. The droplets comprising the oil have an average radius r₀. The fluid comprising the oil has a viscosity μₒ, a surface tension σ₀, and an average concentration in the emulsion C₀. The fluid comprising water has a surface tension σ_{w}. The surface protrusions have an average height h. The droplets comprising the emulsion have an average velocity v when the droplets comprising the emulsion contact the surface.

$$\frac{16 π}{27 {σ}_{o}} * {( \frac{{σ}_{w}}{ρ {R}^{3}} )}^{\frac{1}{2}} * \frac{{r}_{o}^{6}}{{h}^{5}} < 10.$$

$$\frac{20 {C}_{o}^{\frac{3}{2}}}{3 {h}^{\frac{3}{2}}} * {( \frac{{σ}_{o}^{2} {σ}_{w} {μ}_{o}^{2}}{{ρ}^{5}} )}^{\frac{1}{4}} * \frac{1}{{R}^{\frac{3}{4}} {v}^{2 }} > 0.3 .$$

In one embodiment, an article comprises oil disposed on a surface comprising one or more protrusions. The oil is deposited from an emulsion, the emulsion comprises droplets comprising the oil dispersed within a fluid comprising water, and when the droplets comprising the emulsion contact the surface, an average Weber number of the droplets comprising the emulsion is from 45 to 100.

In one embodiment, an article comprises oil disposed on a surface comprising one or more protrusions. The oil is deposited from an emulsion onto the surface when the surface is at least partially covered by oil, the emulsion comprises droplets comprising the oil dispersed within a fluid comprising water, and when the droplets contacted the surface, an average Weber number of the droplets was from 45 to 100.

Certain embodiments relate to compositions and/or kits. In one embodiment, a composition and/or kit comprises a fluid comprising water and a fluid comprising an oil. The fluid comprising water and the fluid comprising the oil are configured to be mixed to form an emulsion to be contacted with a surface comprising one or more protrusions, the emulsion comprises droplets comprising the oil dispersed within the fluid comprising water, and a ratio of an average radius of the droplets comprising the oil to an average height of the protrusions is from 0.01 to 100.

In one embodiment, a composition and/or kit comprises a fluid comprising water and a fluid comprising an oil. The fluid comprising water and the fluid comprising the oil are configured to be mixed to form droplets comprising an emulsion to be contacted with a surface. The droplets comprising the emulsion have an average radius R and an average density ρ. The emulsion comprises droplets comprising the fluid comprising the oil dispersed within the fluid comprising water. The droplets comprising the oil have an average radius r₀. The fluid comprising the oil has a viscosity μ₀, a surface tension σ₀, and an average concentration in the emulsion C₀. The fluid comprising water has a surface tension σ_{w}. The surface comprises one or more protrusions having an average height h. The droplets comprising the emulsion have an average velocity v when the droplets comprising the emulsion contact the surface.

$$\frac{16 π}{27 {σ}_{o}} * {( \frac{{σ}_{w}}{ρ {R}^{3}} )}^{\frac{1}{2}} * \frac{{r}_{o}^{6}}{{h}^{5}} < 10.$$

$$\frac{20 {C}_{o}^{\frac{3}{2}}}{3 {h}^{\frac{3}{2}}} * {( \frac{{σ}_{o}^{2} {σ}_{w} {μ}_{o}^{2}}{{ρ}^{5}} )}^{\frac{1}{4}} * \frac{1}{{R}^{\frac{3}{4}} {v}^{2 }} > 0.3 .$$

In one embodiment, a composition and/or kit comprises a fluid comprising water and a fluid comprising an oil. The fluid comprising water and the fluid comprising the oil are configured to be mixed to form an emulsion to be contacted with a surface, and when the droplets comprising the emulsion contact the surface, an average Weber number of the droplets comprising the emulsion is from 45 to 100.

In one embodiment, a composition and/or kit comprises a fluid comprising water and a fluid comprising an oil. The fluid comprising water and the fluid comprising the oil are configured to be mixed to form an emulsion to be contacted with a surface at least partially covered by oil, and when the droplets contact the surface, an average Weber number of the droplets is from 45 to 100.

Certain embodiments relate to methods. In one embodiment, a method of depositing oil on at least a portion of a surface comprises contacting the surface with an emulsion. The emulsion may comprise droplets comprising an oil dispersed within a fluid comprising water. The surface may comprise one or more protrusions. A ratio of an average radius of the droplets comprising the oil to an average height of the protrusions may be from 0.01 to 100.

In one embodiment, a method of depositing oil on at least a portion of a surface comprises contacting the surface with droplets comprising an emulsion. When the droplets comprising the emulsion contact the surface, an average Weber number of the droplets comprising the emulsion may be from 45 to 100.

In one embodiment, a method of depositing oil on at least a portion of a surface comprises contacting the surface with droplets comprising an emulsion. The droplets comprising the emulsion may have an average radius R and an average density ρ. The emulsion may comprise droplets comprising an oil dispersed within a fluid comprising water. The droplets comprising the oil may have an average radius r₀. The fluid comprising the oil may have a viscosity μ₀, a surface tension σ₀, and an average concentration in the emulsion C₀. The fluid comprising the water may have a surface tension σ_{w}. The surface may comprise one or more protrusions having an average height h. The droplets comprising the emulsion may have an average velocity v when the droplets comprising the emulsion contact the surface. The inequality

$$\frac{16 π}{27 {σ}_{o}} * {( \frac{{σ}_{w}}{ρ {R}^{3}} )}^{\frac{1}{2}} * \frac{{r}_{o}^{6}}{{h}^{5}} < 10$$

may be satisfied. The inequality

$$\frac{20 {C}_{o}^{\frac{3}{2}}}{3 {h}^{\frac{3}{2}}} * {( \frac{{σ}_{o}^{2} {σ}_{w} {μ}_{o}^{2}}{{ρ}^{5}} )}^{\frac{1}{4}} * \frac{1}{{R}^{\frac{3}{4}} {v}^{2 }} > 0.3$$

may be satisfied.

A method may comprise contacting a surface at least partially covered by oil with droplets. The droplets may have an average Weber number from 45 to 100 when they contact the surface.

A method of depositing oil on at least a portion of a surface may comprise determining a target area fraction of the portion of the surface to be covered with oil. The method may also comprise contacting the entirety of the portion of the surface with an emulsion such that, after contact with the emulsion, an area fraction of the portion of the surface covered by oil is within 50% of the target area fraction.

In one embodiment, a method of depositing oil on at least a portion of a surface comprises contacting the entirety of the portion of the surface with an emulsion such that, after contact with the emulsion, an area fraction of the surface is covered by oil. The area fraction of the surface covered by oil after contact with the emulsion is within 50% of an area fraction desired to be covered by oil. The area fraction desired to be covered by oil is from 0 to 1.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1C show one non-limiting embodiment of a method of depositing oil on at least a portion of a surface;

FIG. 2 shows one non-limiting embodiment of a method of contacting a surface at least partially covered by oil with droplets;

FIG. 3 shows a non-limiting embodiment of an article comprising oil disposed on a surface;

FIG. 4A shows one non-limiting embodiment of a surface comprising protrusions;

FIG. 4B shows one non-limiting embodiment of a method of contacting a surface comprising one or more protrusions with droplets;

FIG. 4C shows one non-limiting embodiment of a surface comprising protrusions on which oil is disposed;

FIGS. 5A-5D show DLS measurements of oil droplet size in emulsions, according to certain embodiments;

FIG. 6A shows a schematic depiction of a method of measuring one or more emulsion properties, according to certain embodiments;

FIG. 6B shows optical micrographs of droplets contacting surfaces, according to certain embodiments;

FIG. 6C shows a schematic depiction of interactions between droplets and surfaces comprising protrusions, according to certain embodiments;

FIGS. 7A-7B show micrographs of droplets contacting surfaces, according to certain embodiments;

FIG. 7C is a chart showing the dependence of the ratio of the diameter of the deposit to the maximum diameter as a function of Weber number, according to certain embodiments;

FIG. 7D is a chart showing the dependence of the surface coverage by oil as a function of oil concentration in the emulsion, according to certain embodiments;

FIG. 8 is a chart showing the dependence of the surface coverage by oil as a function of Weber number, according to certain embodiments;

FIG. 9A is a chart showing the dependence of the restitution coefficient of droplets on a surface as a function of Weber number, according to certain embodiments;

FIG. 9B is a chart showing the compositions and Weber numbers of emulsion droplets that bounced and stuck to a surface, according to certain embodiments;

FIG. 10A is a chart showing the normalized contact length as a function of time for droplets contacting a surface, according to certain embodiments;

FIG. 10B is a chart showing the retraction rate as a function of Weber number for droplets contacting a surface, according to certain embodiments;

FIG. 11 shows micrographs of droplets contacting a surface, according to certain embodiments;

FIG. 12A shows a schematic depiction of a droplet contacting a surface comprising protrusions, according to certain embodiments;

FIGS. 12B-12D show micrographs of droplets contacting a surface, according to certain embodiments;

FIG. 12E is a chart showing the diameter of droplets on a surface as a function of time, according to certain embodiments;

FIG. 12F is a chart showing the Weber numbers and oil viscosities of certain droplets that bounced and stuck to surfaces, according to certain embodiments;

FIG. 13A shows micrographs of droplets contacting a surface, according to certain embodiments;

FIG. 13B is a chart showing the retained volume of droplets on a surface as a function of sprayed volume, according to certain embodiments; and

FIG. 13C is a micrograph of a hosta leaf that has been sprayed with water droplets and sprayed with droplets comprising an emulsion.

### DETAILED DESCRIPTION

Methods related to depositing oil on surfaces, methods of contacting surfaces covered by oil with droplets, and associated articles and systems, are generally provided. Certain methods comprise contacting surfaces with emulsions comprising an oil that may deposit onto the surface. Some methods comprise contacting surfaces at least partially covered by oil with droplets. The methods described herein may be particularly advantageous for retaining droplets on surfaces. For example, certain methods that comprise depositing oil onto surfaces may result in the formation of oil deposits that enhance droplet retention on the surfaces. One or more droplets from which the oil deposits onto the surface may be retained on the surface by the oil deposits, and/or the oil deposited from the droplets onto the surface may enhance the ability of the surface to retain further droplets deposited thereon. As another example, certain methods may comprise depositing droplets onto surfaces including one or more features that enhance their retention on those surfaces, such as oil deposits thereon.

In some embodiments, methods comprise depositing oil onto a surface by contacting the surface with an emulsion. The emulsion may have one or more properties that promote the deposition of advantageous amounts of oil therein onto the surface and/or that promote the deposition of oil onto the surface at a rate that is advantageous. The amount of oil deposited may be sufficient to retain droplets on the surface (e.g., of the emulsion, of one or more other fluids). The rate at which the oil is deposited may be sufficient to retain the droplet(s) from which it originated on the surface (e.g., it may occur relatively rapidly, such as over the time period with which the droplet is in contact with the surface before rolling and/or bouncing off). Such properties may include an appropriate ratio of an average radius of oil droplets within the emulsion to an average height of protrusions from the surface, a Weber number in an advantageous range, and/or advantageous relationships between one or more properties of the emulsion.

Some methods comprise depositing oil onto a surface in a controlled manner. In other words, an individual and/or instrument depositing the oil may be capable of depositing the oil in a manner that closely resembles their preferred manner of depositing the oil. As an example, an individual or instrument may be capable of determining a fraction of the surface to be covered by the oil and then depositing the oil to cover a fraction of the surface that is similar to the level they initially determined would be covered by the oil.

Some embodiments relate to articles, compositions, and/or kits. An article composition, and/or kit described herein may be related to a method described herein. By way of example, some articles are products formed by the performance of one or more of the methods described herein. For instance, in some embodiments, an article comprises oil disposed on a surface and is formed according to one or more of the methods described herein. As another example, in some embodiments, a composition and/or a kit is suitable for performing (and/or configured to perform) one or more of the methods described herein. For instance, a composition and/or a kit may comprise one or more components that may be employed in one or more of the methods described herein, such as a fluid comprising an oil and/or a fluid comprising water. In some embodiments, a composition and/or a kit may be configured to be mixed to form an emulsion described herein and/or configured to contact a surface described herein.

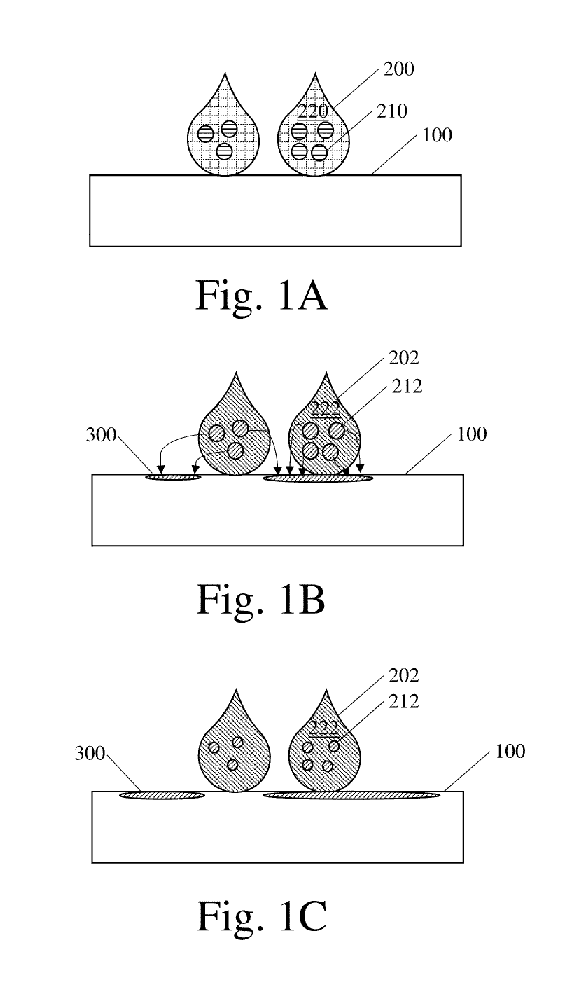

FIGS. 1A-1C show one non-limiting method of depositing oil onto a surface from an emulsion.

In FIG. 1A, a surface 100 is contacted with an emulsion 200 comprising droplets 210 of a first fluid dispersed within a second fluid 220. In some embodiments, the emulsion may also be in the form of droplets. In other words, some embodiments relate to droplets of an emulsion comprising droplets of a first fluid dispersed within a second fluid. Within the emulsion (e.g., within one or more droplets comprising the emulsion), the droplets comprising the first fluid may be arranged in a variety of suitable manners. In some embodiments, the droplets are relatively evenly dispersed throughout the emulsion. In other embodiments, some of the droplets may be clustered in certain areas and/or certain areas may be relatively poor in droplets of the first fluid compared to other areas. The droplets of the first fluid dispersed within the second fluid may, in some cases, not be topologically interconnected.

Some methods relate to oil-in-water emulsions, in which droplets comprising an oil are dispersed within a fluid comprising water (i.e., in which the first fluid comprises an oil and the second fluid comprises water). Some methods relate to water-in-oil emulsions, in which droplets comprising water are dispersed within a fluid comprising an oil (i.e., in which the first fluid comprises water and the second fluid comprises an oil). Other types of emulsions are also contemplated (e.g., emulsions in which a first fluid is dispersed within a second fluid different from the first fluid and/or emulsions in which a first fluid is dispersed within a second fluid within which it is immiscible, either or both of which may comprise a first fluid that comprises water and/or a second fluid that comprises an oil).

Surfaces may be contacted with emulsions in a variety of suitable manners. As described above, in some embodiments, a surface may be contacted with an emulsion by contacting droplets of the emulsion with the surface. The droplets may be sprayed on the surface, dripped on the surface, or contacted with the surface in another suitable manner. Other methods of applying an emulsion to a surface are also contemplated. Such methods may include pouring the emulsion onto the surface, dipping the surface in the emulsion, and the like.

Certain methods comprise depositing oil onto a surface and/or comprise one or more steps that result in the deposition of oil onto a surface. FIG. 1B shows one non-limiting embodiment of a step in which oil is deposited from an emulsion onto a surface. In FIG. 1B, oil from an oil-in-water emulsion deposits onto a surface. In other words, oil from droplets 212 comprising the oil is deposited onto a surface 100 to form oil deposits 300. Droplets 212 comprising the oil are dispersed within a fluid 222 comprising water, both of which are contained within droplet 202. It should be understood that oil may also deposit onto surfaces from water-in-oil emulsions and/or from other types of emulsions. In the case of water-in-oil emulsions, the oil depositing onto the surface would originate from the fluid in which the droplets are dispersed, not the dispersed fluid.

FIG. 1C shows one non-limiting embodiment of a surface onto which oil has been deposited and emulsion droplets thereon. In FIG. 1C, oil from droplets 212 has been deposited onto surface 100 to form oil deposits 300. Droplets 202 comprise a fluid 222 comprising water in which droplets 212 are dispersed. Since some of the oil from droplets 212 has been deposited onto the surface, these droplets are reduced in size. In some embodiments, an appreciable amount fluid from which the oil forming the deposits originates (e.g., droplets comprising the oil) may form the deposit. In such cases, the amount of that fluid in the emulsion may correspondingly decrease (e.g., its volume fraction in the emulsion may decrease). In other embodiments, a relatively small amount of fluid from which the oil forming the deposits originates may form the deposits. In such cases, the amount of that fluid in the emulsion may not appreciably decrease (e.g., its volume fraction in the emulsion may remain substantially the same).

In some embodiments, an emulsion from which oil is deposited onto a surface may be retained on that surface (e.g., embodiments similar to that shown in FIGS. 1A-1C). In some embodiments, an emulsion from which oil is deposited onto a surface may be at least partially removed from that surface (e.g., by bouncing and/or rolling). Some emulsions may be partially retained on the surface and partially removed from the surface. In cases where the emulsion is partially (and/or fully) removed from a surface onto which it deposits oil, subsequent fluids deposited onto the oil-covered surface may be retained on the oil-covered surface.

As shown in FIG. 1C, certain methods comprise depositing oil onto one or more portions of a surface. The portions may be discrete from one another (e.g., not topologically connected with each other), and/or interconnected with one another. In some embodiments, a method may comprise depositing oil onto portions of the surface that are discrete from some other portions of the surface and interconnected with some other portions of the surface. It should be understood that the portions of the surface onto which the oil is deposited may have a variety of suitable sizes and shapes (e.g., circular, fractal, and the like).

Certain methods may comprise depositing oil onto an area fraction of a portion of a surface within a desired range. For instance, a method may comprise determining a target area fraction of the portion of the surface to be covered with oil (in other words, an area fraction of the surface desired to be covered by oil) and contacting the entirety of the portion of the surface with the emulsion. The surface may be contacted with the emulsion in a manner that results in an area fraction of the surface covered by the oil that is relatively close (e.g., within 50%, within 40%, within 30%, within 20%, within 10%, within 5%, within 2%, within 1%) of the target area fraction and/or area fraction of the surface desired to be covered by oil (e.g., after contact with the emulsion). For example, the surface may be contacted with droplets comprising the emulsion. The target area fraction may be an area fraction that promotes droplet retention on the surface (e.g., of droplets comprising the emulsion from which the oil is deposited onto the surface, of other droplets comprising the emulsion, of other droplets). In some embodiments, the target area fraction is an area fraction that promotes droplet retention on the surface without causing excessive and/or wasteful oil deposition onto the surface. The target area fraction may be determined based upon a consideration the average size of droplets comprising the emulsion contacted with the surface, the impact speed of droplets comprising the emulsion contacted with the surface, and/or other parameters. It should be understood that oil may also deposited onto other portions of the surface during this process.

When a method is performed to deposit oil onto at least a portion of the surface, the area fraction of the portion of the surface desired to be covered by oil (i.e., a target area fraction) may have a variety of suitable values. In some embodiments, the area fraction of the portion of the surface desired to be covered by oil is greater than or equal to 0, greater than or equal to 0.05, greater than or equal to 0.1, greater than or equal to 0.15, greater than or equal to 0.2, greater than or equal to 0.25, greater than or equal to 0.3, greater than or equal to 0.35, greater than or equal to 0.4, greater than or equal to 0.45, greater than or equal to 0.5, greater than or equal to 0.55, greater than or equal to 0.6, greater than or equal to 0.65, greater than or equal to 0.7, greater than or equal to 0.75, greater than or equal to 0.8, greater than or equal to 0.85, greater than or equal to 0.9, or greater than or equal to 0.95. In some embodiments, the area fraction of the portion of the surface desired to be covered by oil is less than or equal to 1, less than or equal to 0.95, less than or equal to 0.9, less than or equal to 0.85, less than or equal to 0.8, less than or equal to 0.75, less than or equal to 0.7, less than or equal to 0.65, less than or equal to 0.6, less than or equal to 0.55, less than or equal to 0.5, less than or equal to 0.45, less than or equal to 0.4, less than or equal to 0.35, less than or equal to 0.3, less than or equal to 0.25, less than or equal to 0.2, less than or equal to 0.15, less than or equal to 0.1, or less than or equal to 0.05. Combinations of the above-referenced ranges are also possible (e.g., from 0 to 1). Other ranges are also possible.

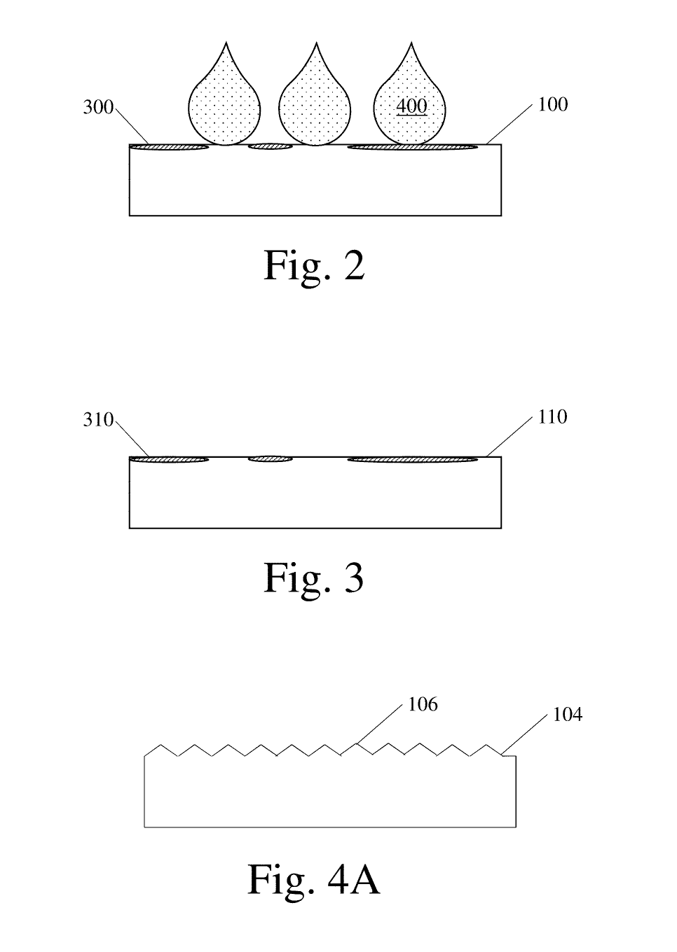

As described above, certain methods comprise contacting a surface at least partially covered by oil with a composition. In some, but not necessarily all, embodiments, the composition comprises droplets. FIG. 2 shows one non-limiting embodiment of a method in which surface 100 partially covered by oil 300 is contacted with droplets 400. The droplets may include droplets comprising emulsions (e.g., they may have one, more, or all of the features described elsewhere herein with respect to droplets comprising emulsions; they may be identical to or similar to the droplets described above with respect to FIGS. 1A-1C), and/or the droplets may include droplets of another type (e.g., droplets comprising water, droplets comprising oil, droplets comprising both oil and water but not an emulsion).

When a method comprises a step of contacting a surface at least partially covered by oil with a composition (e.g., with droplets), the step may be performed at a variety of suitable points in time. In some embodiments, the step may be performed simultaneously with a step of depositing oil onto at least a portion of the surface. For instance, a surface at least partially covered by oil may be contacted with one or more droplets from which oil is further deposited thereon. In such embodiments, the surface may also be contacted with the composition. The composition may comprise droplets from which oil is not deposited thereon (e.g., droplets lacking oil). In some embodiments, the step may be performed after oil is deposited onto the surface from one or more droplets. For example, oil may be deposited onto the surface by contacting the surface with droplets comprising an emulsion and then the partially oil-covered surface may be contacted with the composition. The composition may comprise one or more droplets (e.g., droplets from which oil deposits thereon, droplets from which oil does not deposit thereon). In some embodiments, the step may be performed before at least some oil is deposited onto the surface from one or more droplets. As an example, the surface partially covered by oil may be contacted by the composition and then contacted with one or more droplets that deposit oil thereon. The composition may comprise one or more droplets (e.g., droplets from which oil deposits thereon, droplets from which oil does not deposit thereon).

When a method comprises a step of contacting a surface at least partially covered by oil with a composition (e.g., a composition comprising droplets), the composition contacted with the surface may be at least partially retained on the surface. For instance, the surface may be contacted with droplets, and at least a portion of the droplets may not bounce and/or roll of the surface. In some embodiments, the composition may comprise droplets and a portion of the droplets contacted with the at least partially oil-covered surface may be retained on the surface and at least a portion of the droplets contacted with the at least partially oil-covered surface may not be retained on the surface. Contacting the surface with the droplets may cause at least a portion of the droplets to be retained on the surface.

As described above, some embodiments relate to articles comprising oil disposed on a surface. FIG. 3 shows one non-limiting example of such an embodiment, in which an oil 310 is disposed on a surface 110. The oil disposed on the surface may be oil deposited onto the surface from an emulsion as described elsewhere herein and/or onto a surface as described elsewhere herein. For instance, the emulsion may comprise droplets of oil dispersed within a fluid comprising water and/or having any other characteristic(s) of the emulsions described elsewhere herein. As another example, the oil disposed on the surface may be oil deposited onto a surface comprising one or more protrusions and/or oil deposited onto a surface at least partially covered by oil.

As also described above, certain embodiments relate to an emulsion, a composition comprising one or more components configured to be mixed to form an emulsion, and/or a kit comprising one or more components configured to be mixed to form an emulsion. The emulsion may comprise droplets of a first fluid dispersed within a second fluid. Articles and/or kits may comprise the first fluid and/or the second fluid, and the first and second fluids may be configured to be mixed to form the emulsion. In other words, the article and/or kit may comprise a fluid comprising an oil and/or a fluid comprising water, and the fluid(s) in the article and/or kit may be configured to be mixed together, and/or with other fluids not provided therewith, to form an emulsion. In some embodiments, the first fluid (i.e., the fluid forming droplets dispersed within the second fluid) comprises an oil. Non-limiting examples of suitable oils include pentane, cyclohexane, hexane, heptane, octane, decane, dodecane, tetradecane, hexadecane, silicone oil, BMIm, tetrachloromethane, trichloromethane, dichloromethane, diiodomethane, propane, benzene, toluene, and bromobenzene. In some embodiments, the second fluid (i.e., the fluid in which the droplets are dispersed) comprises water. In other words, the second fluid may be an aqueous fluid.

In some embodiments, an emulsion (e.g., an emulsion in the form of droplets), a droplet, and/or a composition may further comprise one or more additional species. The additional species may be provided with a composition and/or a kit (e.g., as a component of a fluid comprising water, as a component of a fluid comprising an oil, as a further component). The additional species may be an active agent, such as a species that confers a beneficial property onto the emulsion, droplet, and/or composition; and/or a surface on which the droplet, emulsion, and/or composition is disposed (and/or configured to be disposed). Certain beneficial properties may include pest resistance, coloration, flavoring, etc. Non-limiting examples of suitable active agents include agricultural chemicals (e.g., pesticides, herbicides, fertilizers, nutrients), pigments, paints, flavorings, pharmaceutically active ingredients, cosmetics, anti-icing liquids, and fire retardant species. In some embodiments, the active agent may be a pesticide that comprises one or more of dichlorodiphenyltrichloroethane (DDT), hexachlorocyclohexane (HCH), and pentachlorophenol (PCP).

As also described above, certain embodiments relate to contacting surfaces with emulsions, contacting surfaces with droplets (e.g., droplets comprising emulsions, droplets not comprising emulsions), contacting surfaces with compositions (e.g., compositions comprising droplets, compositions comprising an emulsion), and/or compositions and/or kits configured to be contacted with surfaces. In some embodiments, the emulsion, droplet, and/or composition may have one or more beneficial properties, such as one or more properties that aid in emulsion retention, droplet retention, and/or composition retention. In some embodiments, the emulsion, droplet, and/or composition may provide a benefit to the surface contacted with the emulsion, droplet, and/or composition. Examples of these properties will be described in further detail below.

In some embodiments, droplets (e.g., droplets comprising an emulsion) may have an average Weber number that is within a range that is advantageous. As used herein, the Weber number for each droplet is defined by the following equation:

$$We = \frac{ρ {R}_{0} {v}^{2}}{σ} ,$$

where We is the average Weber number, ρ is the average density of the droplets, R₀ is the average radius of the droplets, v is the average velocity of the droplets when they contact the surface, and σ is the average surface tension of the droplets. The average density of the droplets may be determined by weighing a known volume of the droplets, and then dividing the measured mass by the known volume. The average velocity of the droplets when they contact the surface may be determined by impact imaging. The surface tension of the droplets may be determined with a goniometer. The averages above refer to number averages.

Without wishing to be bound by any particular theory, it is believed that droplets that impact surfaces with lower Weber numbers may be prone to bouncing and droplets that impact surfaces with higher Weber numbers may be prone to splashing. Both bouncing and splashing of droplets at the surface may cause the droplets (and/or appreciable fractions thereof) to be removed from the surface. Droplets that impact a surface with intermediate Weber numbers (e.g., Weber numbers between those at which bouncing occurs and those at which splashing occurs) may advantageously be retained on the surface.

Some embodiments comprise contacting droplets (e.g., droplets comprising an emulsion) with a surface that have an average Weber number of greater than or equal to 45, greater than or equal to 50, greater than or equal to 55, greater than or equal to 60, greater than or equal to 65, greater than or equal to 70, greater than or equal to 75, greater than or equal to 80, greater than or equal to 85, greater than or equal to 90, or greater than or equal to 95. Some embodiments comprise contacting droplets with a surface that have an average Weber number of less than or equal to 100, less than or equal to 95, less than or equal to 90, less than or equal to 85, less than or equal to 80, less than or equal to 75, less than or equal to 70, less than or equal to 65, less than or equal to 60, less than or equal to 55, or less than or equal to 50. Combinations of the above-referenced ranges are also possible (e.g., from 45 to 100, or from 50 to 70). Other ranges are also possible.

In embodiments relating to surfaces, a variety of suitable surfaces may be employed. For example, the surface may be a portion of a plant, such as a portion of a leaf, a portion of a root, a portion of a fruit, a portion of a vegetable, and/or a portion of a flower. In some embodiments, the surface may be a portion of a fungus and/or a portion of an insect. In some embodiments, the surface may comprise a portion of a produce item or a surface of a form of vegetation. In certain embodiments, the surface may comprise an edible non-toxic item such as a food item.

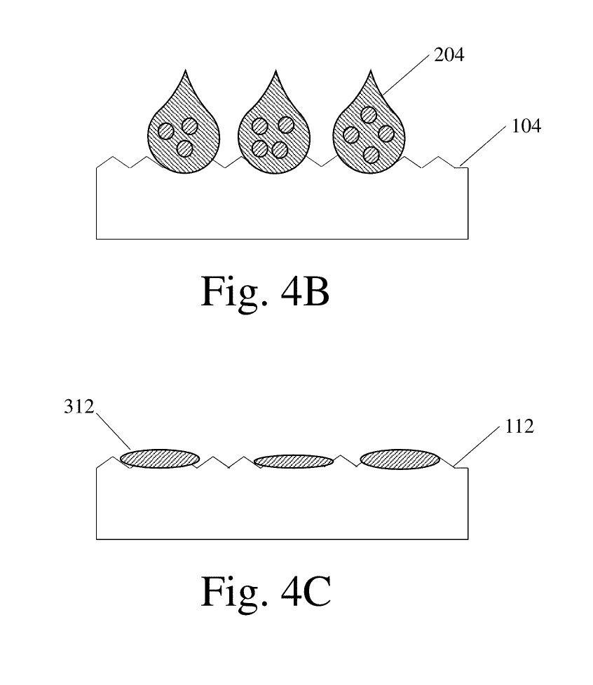

In some embodiments, a surface may comprise one or more protrusions. The protrusions may include any portions of the surface that extend above other portions of the surface. FIG. 4A shows one non-limiting embodiment of a surface 104 comprising protrusions 106, FIG. 4B shows a non-limiting embodiment of a method of contacting a surface 104 comprising protrusions with an emulsion 204, and FIG. 4C shows a non-limiting embodiment of an oil 312 disposed on a surface 112 comprising protrusions. It should be understood that the protrusions shown in FIGS. 4A, 4B, and 4C are merely exemplary, and that surfaces contemplated herein may comprise protrusions that differ in one or more ways from the protrusions shown therein. In some embodiments, the protrusions may be relatively uniform (e.g., in size, shape, and/or spacing). In some embodiments, the protrusions may differ across the surface in one or more ways. It should be understood that the protrusions, if present, may have a variety of suitable sizes, shapes, aspect ratios, spacings, and the like. The protrusions may extend directly above the surface, may extend at an angle from the surface, and/or may overhang other portions of the surface.

When a surface comprises protrusions, species disposed on the surface may interact with the protrusions in a variety of suitable manners. In some embodiments depositing oil onto a surface comprises depositing oil between two or more protrusions thereon. In other words, in some embodiments oil deposited onto a surface may penetrate between two or more protrusions thereon and/or fill in depression(s) between two or more protrusions thereon. In some embodiments, depositing oil onto a surface comprises depositing oil onto the surface such that it does not penetrate into any of, or does not penetrate into a portion of, the spaces between the protrusions thereon. Similarly, some embodiments relate to oil disposed on a surface that is positioned between two or more protrusions thereon (i.e., filling in depression(s) between two or more protrusions thereon) and some embodiments relate to oil disposed on a surface that does not penetrate into any of, or does not penetrate into a portion of, the spaces between the protrusions on thereon.

It should also be understood that certain embodiments may comprise contacting surfaces comprising protrusions with compositions other than emulsions (e.g., droplets lacking emulsions, other fluids lacking emulsions) and/or compositions in forms other than droplets (e.g., emulsions not in the form of droplets, other fluids not in the form of droplets).

Droplets (e.g., droplets comprising an emulsion) applied to surfaces comprising one or more protrusions may interact with the protrusions in an advantageous manner. In some embodiments, certain relationships between one or more properties of the droplet and one or more properties of the protrusions may facilitate beneficial interactions. For instance, for emulsions comprising droplets comprising oil dispersed in a second fluid (e.g., a fluid comprising water), certain ratios of the average radius of the droplets comprising the oil to the average height of the protrusions may be advantageous. Such ratios may promote deposition of oil onto the surface in a manner that is controllable, covers an advantageous amount of the surface, and/or deposits onto the surface at a rate that is beneficial for droplet retention (e.g., retention of the droplet comprising the emulsion).

The ratio of the average radius of the droplets comprising the oil to the average height of the protrusions may be greater than or equal to 0.01, greater than or equal to 0.02, greater than or equal to 0.05, greater than or equal to 0.1, greater than or equal to 0.2, greater than or equal to 0.5, greater than or equal to 1, greater than or equal to 2, greater than or equal to 5, greater than or equal to 10, greater than or equal to 20, or greater than or equal to 50. The ratio of the average radius of the droplets comprising the oil to the average height of the protrusions may be less than or equal to 100, less than or equal to 50, less than or equal to 20, less than or equal to 10, less than or equal to 5, less than or equal to 2, less than or equal to 1, less than or equal to 0.5, less than or equal to 0.2, less than or equal to 0.1, less than or equal to 0.05, or less than or equal to 0.02. Combinations of the above-referenced ranges are also possible (e.g., from 0.01 to 100). Other ranges are also possible. The averages above refer to number averages. The average radius of the droplets comprising the oil may be determined by dynamic light scattering. The average height of the protrusions may be determined by atomic force microscopy.

In some embodiments, droplets (e.g., droplets comprising an emulsion) may have certain properties that relate to each other and to a surface with which they are contacted in advantageous ways. For droplets comprising emulsions comprising droplets comprising an oil dispersed in a fluid comprising water, one such way is summarized by Inequality 1:

$$\frac{16 π}{27 {σ}_{o}} * {( \frac{{σ}_{w}}{ρ {R}^{3}} )}^{\frac{1}{2}} * \frac{{r}_{o}^{6}}{{h}^{5}} < X , \qquad ( Inequality 1 )$$

where σ₀ is the surface tension of the fluid comprising the oil, σ_{w} is the surface tension of the fluid comprising the water, ρ is the average density of the droplets comprising the emulsion, R is the average radius of the droplets comprising the emulsion, r₀ is the average radius of the droplets of the fluid comprising the oil, and h is the average height of the surface protrusions. The surface tension of the fluid comprising the oil may be determined with a goniometer. The surface tension of the fluid comprising the water may be determined with a goniometer. The average density of the droplets comprising the emulsion may be determined by weighing a known volume of the droplets comprising the emulsion, and then dividing the measured mass by the known volume. The average velocity of the droplets when they contact the surface may be determined by impact imaging. The average radius of the droplets comprising the emulsion may be determined by image analysis. The average radius of the droplets of the fluid comprising the oil may be determined by image analysis. The average height of the surface protrusions may be determined by atomic force microscopy. The averages above refer to number averages.

In some embodiments, X in Inequality 1 is 10, 7.5, 5, or 2. Other values are also possible.

As another example, in some embodiments droplets comprising emulsions comprising droplets comprising an oil dispersed in a fluid comprising water may satisfy Inequality 2:

$$\frac{20 {C}_{o}^{\frac{3}{2}}}{3 {h}^{\frac{3}{2}}} * {( \frac{{σ}_{o}^{2} {σ}_{w} {μ}_{o}^{2}}{{ρ}^{5}} )}^{\frac{1}{4}} * \frac{1}{{R}^{\frac{3}{4}} {v}^{2 }} > Y , \qquad ( Inequality 2 )$$

where C₀ is the average concentration of the fluid comprising the oil in the emulsion, h is the average height of the surface protrusions, σ₀ is the surface tension of the fluid comprising the oil, σ_{w} is the surface tension of the fluid comprising the water, μ₀ is the viscosity of the fluid comprising the oil, ρ is the average density of the droplets comprising the emulsion, R is the average radius of the droplets comprising the emulsion, and v is the average velocity of the droplets comprising the emulsion when they contact the surface. The average concentration of the fluid comprising the oil in the emulsion may be determined by measuring the volume fractions of each liquid forming the emulsion during emulsion preparation and equating the measured volume fraction of the oil with the average concentration of the oil in the emulsion. The surface tension of the fluid comprising the oil may be determined with a goniometer. The surface tension of the fluid comprising the water may be determined with a goniometer. The viscosity of the fluid comprising the oil may be determined with a viscometer. The average density of the droplets comprising the emulsion may be determined by weighing a known volume of the droplets comprising the emulsion, and then dividing the measured mass by the known volume. The average radius of the droplets comprising the emulsion may be determined by image analysis. The average velocity of the droplets when they contact the surface may be determined by impact imaging. The averages above refer to number averages.

In some embodiments, Y in Inequality 2 is 0.3, 0.5, 0.75, 1, 1.3, 1.5, 1.75, or 2. Other values are also possible.

A surface as described herein may have a variety of suitable roughnesses. The roughness of the surface may be greater than or equal to 1 nm, greater than or equal to 2 nm, greater than or equal to 5 nm, greater than or equal to 10 nm, greater than or equal to 20 nm, greater than or equal to 50 nm, greater than or equal to 100 nm, greater than or equal to 200 nm, greater than or equal to 500 nm, greater than or equal to 1 micron, greater than or equal to 2 microns, greater than or equal to 5 microns, greater than or equal to 10 microns, greater than or equal to 20 microns, greater than or equal to 50 microns, greater than or equal to 100 microns, or greater than or equal to 200 microns. The roughness of the surface may be less than or equal to 500 microns, less than or equal to 200 microns, less than or equal to 100 microns, less than or equal to 50 microns, less than or equal to 20 microns, less than or equal to 10 microns, less than or equal to 5 microns, less than or equal to 2 microns, less than or equal to 1 micron, less than or equal to 500 nm, less than or equal to 200 nm, less than or equal to 100 nm, less than or equal to 50 nm, less than or equal to 20 nm, less than or equal to 10 nm, less than or equal to 5 nm, or less than or equal to 2 nm. Combinations of the above-referenced ranges are also possible (e.g., from 1 nm to 500 microns, or from 20 nm to 50 microns). Other ranges are also possible. The roughness of the surface may be determined by atomic force microscopy.

A surface may have a variety of suitable contact angles with water (e.g., prior to deposition of oil thereon, prior to contact with droplets, prior to contact with droplets comprising an emulsion, prior to contact with droplets comprising an emulsion). The water contact angle of the surface may be greater than or equal to 90°, greater than or equal to 100°, greater than or equal to 110°, greater than or equal to 120°, greater than or equal to 130°, greater than or equal to 140°, greater than or equal to 150°, greater than or equal to 160°, or greater than or equal to 170°. The water contact angle of the surface may be less than or equal to 180°, less than or equal to 170°, less than or equal to 160°, less than or equal to 150°, less than or equal to 140°, less than or equal to 130°, less than or equal to 120°, less than or equal to 110°, or less than or equal to 100°. Combinations of the above-referenced ranges are also possible (e.g., from 90° to 180°). Other ranges are also possible. The water contact angle of the surface may be determined by a goniometer.

Certain methods described herein result in the retention of large volumes of fluid on surfaces (e.g., large volumes of an emulsion, large volumes of droplets comprising an emulsion, large volumes of droplets, large volumes of compositions). In some embodiments, performing a method as described herein (e.g., contacting a surface with an emulsion, contacting a surface with droplets, contacting a surface with droplets comprising an emulsion, contacting a surface with a composition) causes the surface to hold greater than or equal to greater than or equal to 0.5 mL/cm² of a fluid, greater than or equal to 1 mL/cm² of a fluid, greater than or equal to 2 mL/cm² of a fluid, greater than or equal to 4 mL/cm² of a fluid, greater than or equal to 10 mL/cm² of a fluid, greater than or equal to 20 mL/cm² of a fluid, or greater than or equal to 40 mL/cm² of a fluid. Performing a method as described herein may cause the surface to hold less than or equal to 100 mL/cm² of a fluid, less than or equal to 40 mL/cm² of a fluid, less than or equal to 20 mL/cm² of a fluid, less than or equal to 10 mL/cm² of a fluid, less than or equal to 4 mL/cm² of a fluid, less than or equal to 2 mL/cm² of a fluid, or less than or equal to 1 mL/cm² of a fluid. Combinations of the above-referenced ranges are also possible (e.g., from 0.5 mL/cm² of a fluid to 100 mL/cm² of a fluid, or from 4 mL/cm² of a fluid to 40 mL/cm² of a fluid). Other ranges are also possible. The fluid held by the surface may be determined by determining the area of the surface by image analysis, weighing the surface both prior to and after to performing the method, and then dividing the increase in weight after performing the method by the area of the surface.

In some embodiments, performing a method as described herein comprising contacting a surface with a composition (e.g., contacting a surface with an emulsion, contacting a surface with droplets, contacting a surface comprising droplets comprising an emulsion) causes greater than or equal to 30% of the surface exposed to the composition to be covered by the composition, greater than or equal to 40% of the surface exposed to the composition to be covered by the composition, greater than or equal to 50% of the surface exposed to the composition to be covered by the composition, greater than or equal to 60% of the surface exposed to the composition to be covered by the composition, greater than or equal to 70% of the surface exposed to the composition to be covered by the composition, greater than or equal to 80% of the surface exposed to the composition to be covered by the composition, or greater than or equal to 90% of the surface exposed to the composition to be covered by the composition. In some embodiments, performing a method as described herein comprising contacting a surface with a composition causes less than or equal to 100% of the surface exposed to the composition to be covered by the composition, less than or equal to 90% of the surface exposed to the composition to be covered by the composition, less than or equal to 80% of the surface exposed to the composition to be covered by the composition, less than or equal to 70% of the surface exposed to the composition to be covered by the composition, less than or equal to 60% of the surface exposed to the composition to be covered by the composition, less than or equal to 50% of the surface exposed to the composition to be covered by the composition, or less than or equal to 40% of the surface exposed to the composition to be covered by the composition. Combinations of the above-referenced ranges are also possible (e.g., from 30% to 100%). Other ranges are also possible. The percentage of the surface exposed to the composition that is covered by the composition may be determined by photography and image analysis.

In some embodiments, a composition and/or a kit may be provided with directions for use. The directions for use may describe how to employ the composition and/or kit to deposit oil on at least a portion of a surface. By way of example, the directions for use may comprise instructions for how to perform any of the methods described herein and/or for how to form any of the articles described herein. In some embodiments, the directions for use describe procedures for mixing the component(s) of the composition and/or kit with each other and/or other components not provided therewith. As another example, the directions for use may describe directions for depositing an emulsion formed by the composition and/or kit (and/or one or more components thereof) on a surface. As further examples, the directions for use may comprise storage instructions and/or instructions for assessing the quality of emulsions and/or articles produced by the composition and/or kit. The directions for use may describe further components not provided with the composition and/or kit that may be added thereto, such as further fluids (e.g., a fluid comprising water), additives, and/or other suitable components.

The following examples are intended to illustrate certain embodiments of the present invention, but do not exemplify the full scope of the invention.

### EXAMPLE 1

This Example describes the deposition of emulsions with enhanced utility for droplet retention on surfaces. The effects of different parameters of the emulsion on droplet retention are discussed.

### Introduction

When certain emulsions are deposited onto a hydrophobic surface, they may bounce or roll off the surface. Hydrophobic surfaces are common in agriculture; many plants are hydrophobic, possibly due to the presence of waxes and hairs on the surface of their leaves. Hydrophobic surfaces may also be present on metal strips, onto which emulsions may be sprayed during manufacturing of metal strips to provide cooling and/or lubrication. In this Example, emulsion designs in which droplet size, oil fraction and oil viscosity are selected to allow sprayed droplets to stick on hydrophobic surfaces are presented. Depositing such emulsions on a surface may cause an oil layer and a nanometric oil ridge to be formed on the surface (e.g., in-situ) around the impacting droplets. Either or both of these features may create an attractive force that may prevent emulsion droplets them from bouncing upon impact. Another aspect is a method of spraying where the overall droplet size and impact velocity of such emulsion droplets are tuned to allow them to stick upon impact.

Some emulsions in this Example are deposited onto liquid-infused surfaces (LIS). LIS include textured surfaces that are coated with a layer of oil that wicks into their texture and is held there by capillary forces. These surfaces may reduce fluid adhesion and/or may increase droplet mobility. LIS may find applications in container coatings, drag reduction, anti-icing, heat transfer enhancements, and the like.

Certain droplets (e.g., emulsions, liquids including one species, liquids comprising mixtures of two or more species) described herein may be retained on LB.

This Example also presents partially liquid-infused surfaces, in which oil impregnates the surface in various patches while leaving other portions of the surface free from oil. Some partially liquid-infused surfaces may create a normal attractive force that enhances droplet sticking on impact. Some partially liquid-infused surfaces may be less slippery than fully liquid-infused surfaces. Less slippery surfaces may, in some cases, be useful in applications where capture of droplets is sought.

This Example also discusses methods of making partially liquid-infused surfaces and methods of tuning the fraction of oil on partially liquid-infused surfaces. In certain embodiments, tuning the fraction of oil on partially liquid-infused surfaces may tune the slip and/or adhesion of liquids on the partially liquid-infused surface.

When droplets are sprayed onto surfaces under certain conditions, oil deposits are left on the surface after impacting droplets bounce off. These deposits can take on a variety of structures, including full oil layers and patchy oil deposits including many small “islands” of oil. In certain cases, droplets may be sprayed onto surfaces such that no deposits may be formed. This Example includes modeling of deposit formation and identification of certain combinations of parameters that result in advantageous deposit formation. One experimental technique employed in this Example is impacting droplets with certain oil concentrations and viscosities on inclined hydrophobic surfaces such that the droplets only impact the same spot once. This technique may be employed to tune coverage of the hydrophobic surface with the droplet. It may, in some cases, be employed to make surfaces that are partially or completely infused with oil, and/or surfaces with a controlled amount of oil coverage.

Upon impact, some emulsions bounce and some emulsions stick. It was found that having a partially infused surface or a complete LB can generate a suction force that may prevent droplets from bouncing. Values of oil concentration and emulsion viscosity that successfully arrest droplets with a certain size and impact velocity were found. Different regimes of interaction of droplets with LIS were identified, including an impact velocity regime (onset of splashing) where the restitution coefficient (the energy fraction that the droplet has to bounce) sharply drops. It was found that droplets that impact surfaces in this regime have advantageously high retention on those surfaces.

The kinetics of the impregnation of the surface by oil droplets from the emulsion during the impact timescale was also studied. It was found that certain ranges of viscosities and oil droplet sizes promote advantageous oil impregnation and prevention of bouncing in the impact timescale.

### Results

### Emulsion Impacts on Non-Wetting Surfaces

This Example describes results from a study on the impacts of emulsion droplets on superhydrophobic surfaces. It is found that the impact behavior can vary in a non-intuitive way as a function of the Weber number. Droplets can bounce at low We, stick at moderate We, as the contact line starts destabilizing at the onset of splashing, and/or bounce at high We. It is found that the surface may become impregnated in-situ by the oil droplets in the emulsion during the spreading phase and that during the retraction, droplets can effectively see a lubricant impregnated surface. It is shown that the restitution coefficient may decrease (e.g., sharply) at the onset of splashing. This decrease in kinetic energy may cause viscous dissipation to balance the kinetic energy, and/or may prevent the droplet from bouncing. At higher We, the kinetic energy may increase, and may do so to an extent that causes the droplet to bounce. In some cases, viscosity may play two conflicting roles: it increases the dissipation in the lubricating film and it may increase the timescale of impregnation. In some cases, high viscosity oils may not be effective at the timescale of a droplet impact.

### Methods and Materials

### Emulsion Preparation

The oils used here were hexadecane and silicone oils of various viscosities. Emulsions were prepared in 10 mL batches, using a micropipette to add the appropriate volume of water and oil for a certain concentration in a vial. No surfactants were added. The solution was then mixed with a probe sonicator (Sonics Vibra Cell VCX 750) for 90 seconds at 60% power. Emulsions were used in the 30 minutes following preparation.

### Surface Preparation

Superhydrophobic nanograss surfaces were fabricated using Reactive Ion Etching with O₂ and SF₆ on Silicon substrates. The resulting surface had a random texture with features on the order of 200 nm, and was superhydrophilic. The surface was then coated with Octadecyltrichlorosilane (OTS), a hydrophobic modifier, to make it superhydrophobic. The advancing contact angle of DI water on this surface was 165°±2° and the receding angle was 160 °±3°. Angles were measured with a Ramé-hart model 500 goniometer at room temperature.

Transparent superhydrophobic surfaces were made by dip-coating (at a speed of 10 mm/min) glass slides in a dispersion of OTS-coated silica nanoparticles of average size 12 nm (size range 8-15 nm). The dispersion was prepared by mixing 1 wt % nanoparticles in ethanol for 2 minutes with probe sonication at 60% power.

### LIS Preparation

Liquid-impregnated surfaces were made by dip-coating OTS-coated surfaces in oil solutions. Both nanograss and surfaces with micro-texture (square posts with 10 micron width, 10 micron pitch and 21 micron height) were used. The withdrawal speed in the dip-coating process was kept lower than

$${V}_{crit} = 0.12 {μ}_{0} {σ ( \frac{t}{{l}_{c}} )}^{3 / 2} ,$$

where μ₀ is the viscosity of the oil, σ its surface tension, l_{c} the capillary length, and t the height of the texture. Without wishing to be bound by any particular theory, it is believed that withdrawal speeds in this range may enhance conformal coverage of the oil on the surface.

### Emulsion Size Determination

The size of oil droplets in the emulsion was measured with dynamic light scattering (DLS) at room temperature. DLS measurements were performed using DynaPro NanoStar, capable of measuring droplets with radii in the 0.2 nm-2.5 micron range. DLS measurements were acquired 10 times for each sample.

### Coverage Determination

Emulsion impacts were performed on a slightly inclined surface (10°). Without wishing to be bound by any particular theory, it is believed that inclined surfaces reduce the probability that droplets deposited thereon rebound on the same spot. The original impact spot was then observed under an optical microscope (Zeiss Axio Zoom.V16). Areas impregnated with oil appeared to be colored, possibly due to interference color effects (their thickness were in the range of the hundreds of nanometers). Image processing using the software ImageJ was then used to calculate the fraction of the area that oil occupied.

### Physical Properties of the Oils Employed

| | | Silicone | Silicone | Silicone | Silicone |
|---|---|---|---|---|---|
| | | oil with a | oil with a | oil with a | oil with a |
| | | viscosity | viscosity | viscosity | viscosity |
| | Hexa- | of 1.5 | of 10 | of 100 | of 1000 |
| | decane | Pa*s | Pa*s | Pa*s | Pa*s |
| Viscosity | 3 | 1.5 | 10 | 100 | 1000 |
| (10⁻³ Pa*s) | | | | | |
| Surface | 27 | 18 | 20 | 20.9 | 21.2 |
| tension | | | | | |
| (mN*m⁻¹) | | | | | |
| Density | 773 | 851 | 935 | 965 | 970 |
| (kg*m⁻³) | | | | | |

### Results and Mechanism

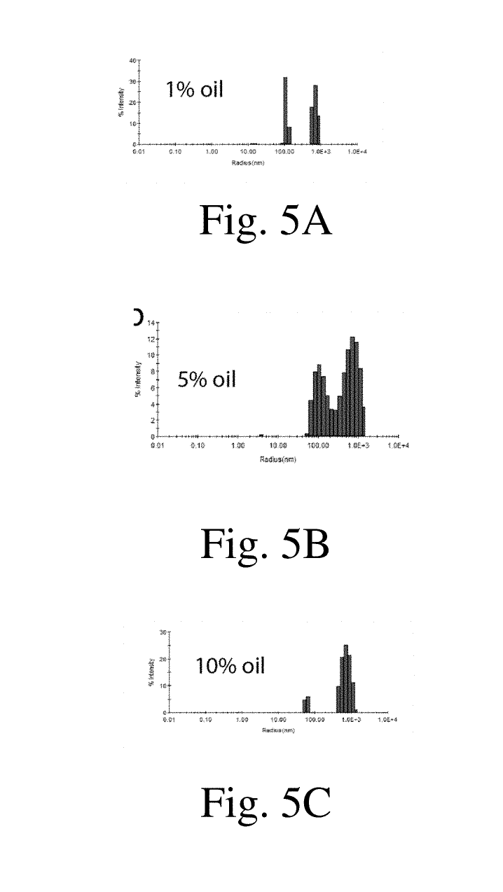

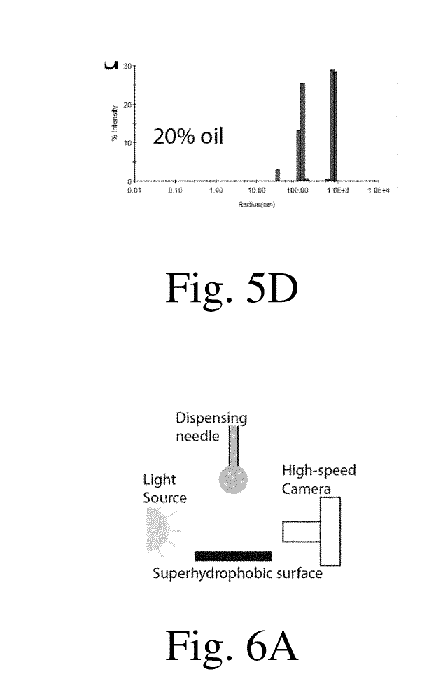

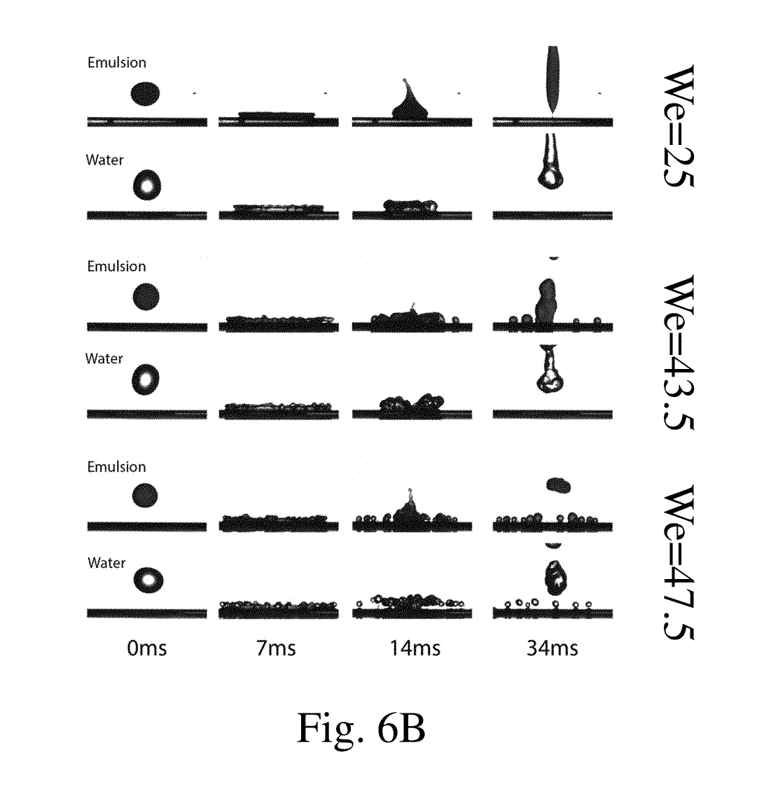

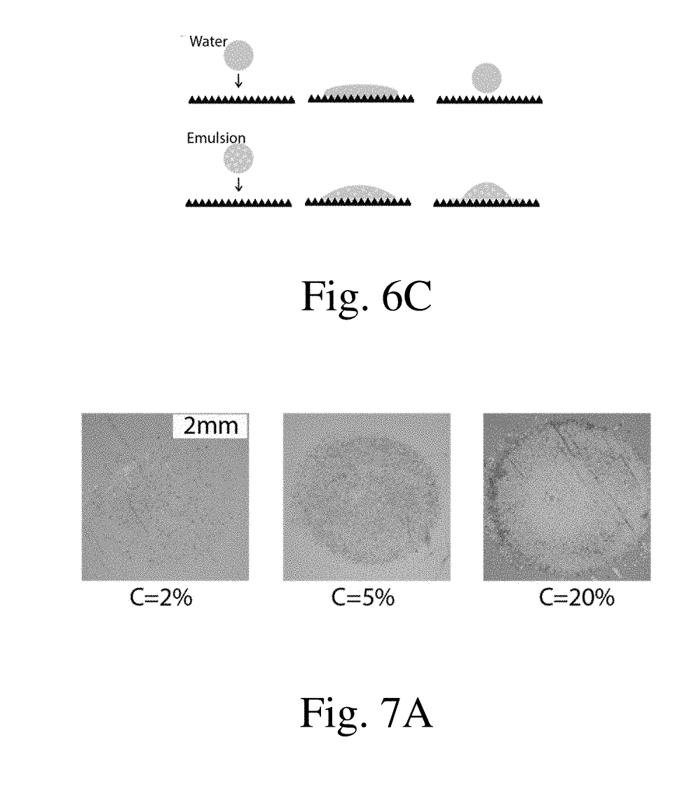

A mixture of hexadecane (oil viscosity μ₀˜3 cSt) with water was sonicated using a probe sonicator to produce an oil-in-water emulsion of average oil droplet size ˜700 nm (FIGS. 5A-5C). No surfactants were added. The experimental setup is shown in FIG. 6A. A dispensing needle placed at an adjustable height was connected to a syringe filled with the emulsion. Droplets were dispensed one at a time and fell by gravity to impact a superhydrophobic surface (165° contact angle, with 5° hysteresis). The impact was imaged with a high speed camera at 10,000 fps with back lighting. The parameters that were varied were the oil concentration in the emulsion, the size of the droplet (by changing the needle) and the impact speed (by changing the height).

FIG. 6B shows micrographs of droplets with a radius of 2 mm and oil concentration of 10% impacting the surface. At a Weber number of 25, both emulsion droplets and water droplets expand, retract and bounce similarly to water droplets on the same surface. The bouncing is slightly delayed for the emulsion droplet in comparison to the water droplet, suggesting there may be some energy dissipation. At a Weber number of 43.5, water droplets bounce while emulsion droplets stick to the surface. In some cases, the sticking coincides with the onset of splashing in the impacting droplet. When the Weber number is increased beyond 43.5, water droplets splash and bounce and emulsion droplets start bouncing again.

For certain emulsion droplets, there is a bouncing-sticking-bouncing transition. The sticking window may coincide with the onset of splashing.

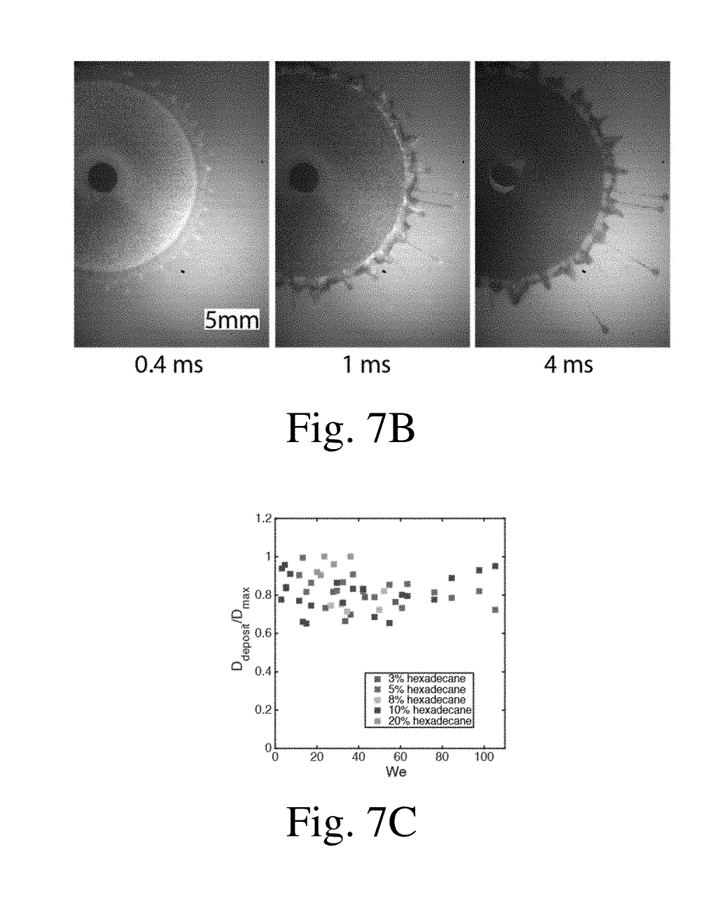

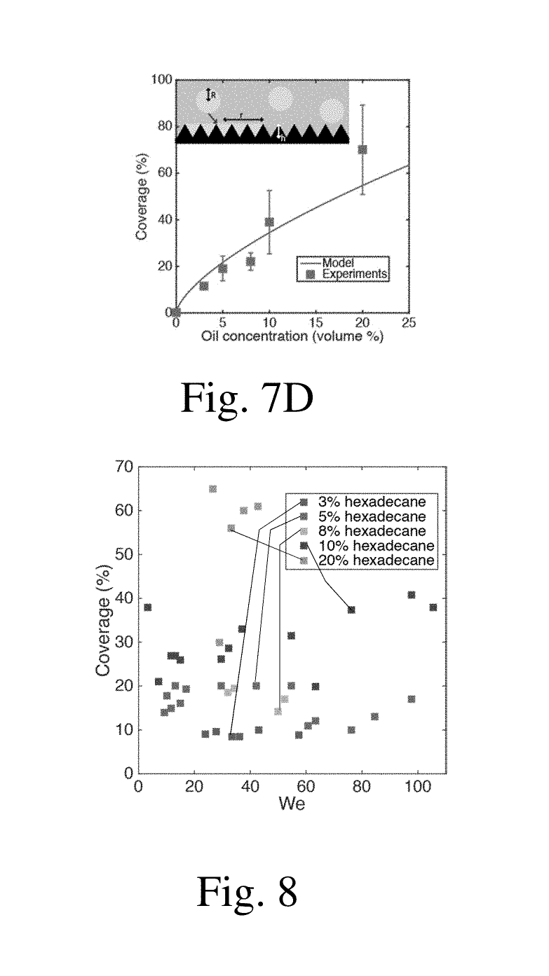

Without wishing to be bound by any particular theory, it is believed that when emulsion droplets impact the surface, some of the oil droplets within the emulsion droplets come to the surface and impregnate its texture. As the droplet starts retracting, it may not retract on a superhydrophobic surface in a Cassie state. Instead, the droplet may retract on a partially oil-impregnated surface (FIG. 6C). If an oil layer is formed in-situ under the droplet, it may promote viscous dissipation and/or inhibit bouncing. The surface was imaged after emulsion impact under an optical microscope. To reduce the likelihood of multiple rebounds on the same spot, experiments were performed on a slightly inclined surface (˜10°). FIG. 7A shows microscope images of the surface after emulsion impacts at different concentrations. Oil deposits can be seen and it can be observed that oil is deposited quite uniformly across the area under the droplet. In some cases, the amount of oil deposited on the surface increases with the concentration of the oil in the emulsion. A droplet impact was imaged on a transparent superhydrophobic surface from the bottom. The lens was focused exactly on the surface. The images are shown in FIG. 7B, where it can be seen that oil droplets are touching the surface during the spreading phase. The oil deposits remain on the surface as the droplet retracts, showing that, in some cases, the droplet retracts on a partially oil-impregnated surface.

To quantify the impregnation of oil, droplet impact experiments were repeated with various concentrations, radii and velocities. For each experiment, the diameter D_{deposit} of the deposit left on the surface was measured from microscope images, and compared to the maximum expansion diameter Dₘₐₓ of the expanding droplet (from high-speed movies). FIG. 7C shows that the ratio

$$\frac{{D}_{deposit}}{{D}_{ma x}}$$

was around 1 for all Weber numbers and all oil concentrations. This may suggest that oil deposits on the surface in all locations where the emulsion is in contact with the surface. The coverage φ of the surface by oil, defined as the area covered by oil divided by the total area under the droplet, was also measured. The coverage values showed some scattering, but no trend was observed as the Weber number was varied (FIG. 8). Average values of the coverage are shown in FIG. 7D as a function of oil concentration.

The observed distribution of the oil residue is fairly uniform across experiments with different Weber numbers and covers the whole area under the droplet, and so it is possible that the impregnation of oil droplets on the surface is not driven by internal flow in the droplet. One possibility is that contact between oil droplets that are located close to the surface with the solid texture may initiate the impregnation. Deceleration-driven motion of oil droplets may not explain the behavior either, because it is believed that less dense oil droplets could tend to go upwards rather than to the surface. Moreover, since diffusion timescales of oil droplets in the water droplets is much slower than the contact time (diffusion length in timescale of impact is ˜100 nm), for the purposes of this Example, it is assumed that only half (moving downwards) of the oil droplets located in the first layer of fluid contacting the surface will touch the surface during the impact.

To simplify the calculations, a uniform distribution of the oil droplets where the oil droplets form an array with a fixed distance between droplets is assumed. In this scenario, the line concentration of droplets C₁ can be approximated as the diameter of an oil droplet divided by the center-to-center distance between two droplets. The area concentration, or how much of a certain plane is occupied by oil is then Cₐ=Cₗ². The volume concentration, which is the quantity fixed in the experiments is Cᵥ=Cₗ³.

The amount of oil that is in the closest layer of fluid can be determined by the area concentration of oil there Cₐ=Cᵥ^{2/3}.

When an individual oil droplet of radius R in that layer impregnates the texture of the surface, of height h, it spreads in a circular layer of radius r. (Inset of FIG. 7D)

### Conservation of Volume Gives:

4/3πR³=πr²h

### Thus

$$r = \sqrt{\frac{4}{3} \frac{R}{h}} R$$

As it impregnates the surface, each droplet will occupy a radius r, instead of R when it was in bulk. Thereby, assuming all oil droplets in the first layer impregnate the surface, the area coverage will be

$$ϕ = \frac{1}{2} {{C}_{a} ( \frac{r}{R} )}^{2} = \frac{2}{3} \frac{R}{h} {C}_{v}^{2 / 3} .$$

For emulsions with oil droplets of 350 nm radius, and a nanograss surface with a texture height of about 150 nm,

$$\frac{2}{3} \frac{R}{h} \sim 1.6$$

and the coverage will be φ˜1.6 Cᵥ^{2/3}.

FIG. 7D shows the theoretical model curve as well as experimental measurements of the coverage for various concentrations. For the experiments performed here, the model can accurately predict the coverage with no fitting parameters.

### Model for Sticking of Emulsion Droplets at Onset of Splashing

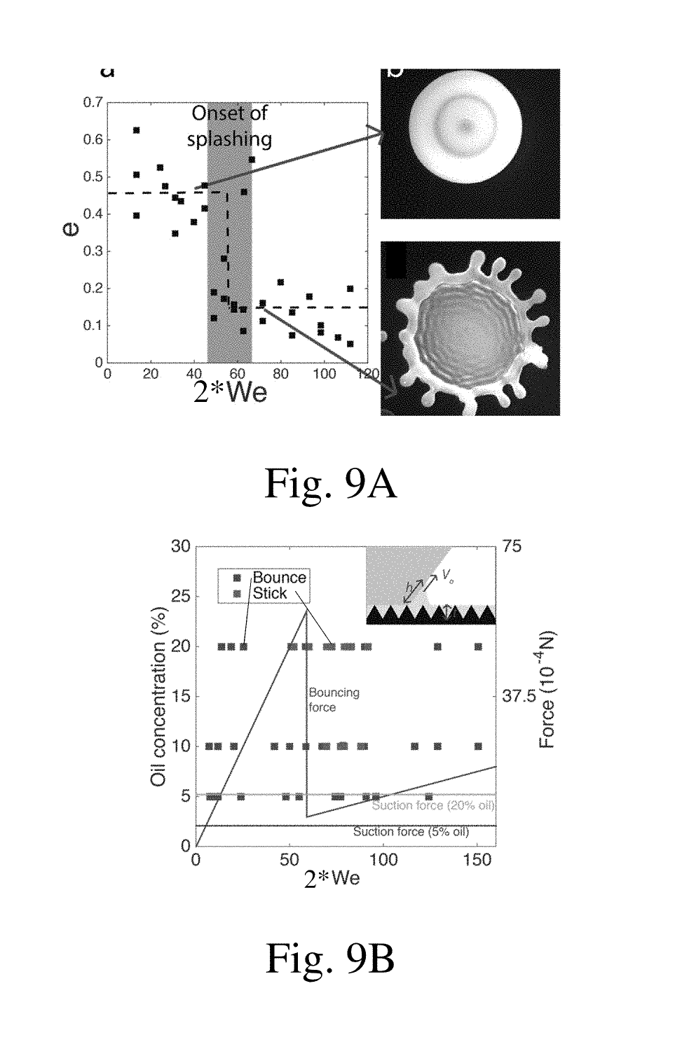

The droplet impinges with a kinetic energy E_{k,i}=4/3πR₀³ρv². During the impact some of this energy can be dissipated by viscosity and when the droplet bounces off, some of its initial kinetic energy is converted into internal vibration energy. The amount of remaining energy for bouncing can be estimated by measuring the restitution coefficient e₀ in the cases where droplets of water bounce (FIG. 9A). The restitution coefficient is the ratio of the vertical momentum after rebound to the momentum before impact. For moderate We (We<25), the droplet retracts as a whole axisymmetrically (FIG. 9A) and e₀˜0.45. The remaining kinetic energy of the droplet is then Eₖ=e₀²E_{k,i}˜0.2 4/3πR₀³ρv²˜0.84. R₀³ρv².

At a Weber number between 25 and 35, the transition to splashing occurs (FIG. 9A). Part of the kinetic energy is then expelled sideways into the smaller splashing droplets The restitution coefficient sharply drops then by a factor 3 and the kinetic energy then becomes

Eₖ=e₀²E_{k,i}˜0.09.R₀³ρv²

From these estimates of the bouncing kinetic energy, the force the droplet experiences as it bounces can be estimated.

The droplet goes from a zero vertical velocity to the bouncing velocity corresponding to that kinetic energy over a typical distance R₀, the radius of the droplet. Therefore the bouncing force is equal to the acceleration the droplet experiences:

$${F}_{b} \sim \frac{{E}_{k}}{{R}_{0}} .$$

As the droplet retracts, it sees a surface that is partially filled with oil. The oil layer under the droplet exerts a suction force on the droplet that can prevent it from bouncing. The droplet will stick if the suction force from the oil layer is high enough to balance the bouncing force F_{b} exerted on the droplet due to its inertia.

In this model, viscous dissipation in the water phase is neglected. As oil spreads on the droplet to start forming the ridge observed at the contact line of droplets on LIS, it generates a capillary suction force due to the Laplace pressure difference across the concave ridge, whose radius of curvature is small enough to make this force non-negligible. When this oil suction overcomes the bouncing force, bouncing is suppressed. The suction pressure is approximately

$${P}_{s} \sim \frac{{σ}_{o}}{h}$$

where σ₀ is tne surface tension of the oil and h is the size of the ridge (FIG. 9B).

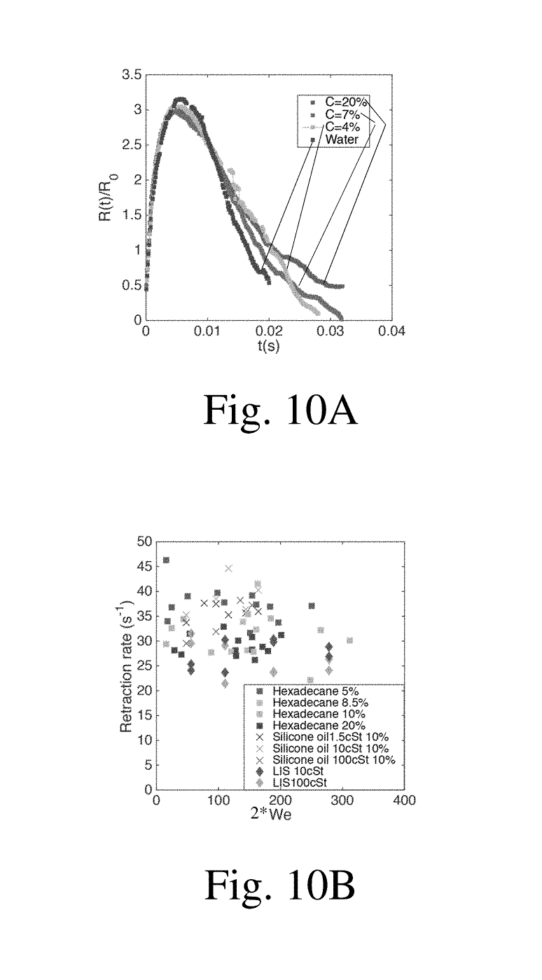

This dissipation mechanism is agreement with the observation that the retraction rates of emulsion droplets as well as the retraction rates of water droplets on liquid-impregnated surfaces (with viscosities up to 100 cSt) are not affected by the oil viscosity. FIG. 10A shows the time evolution of the contact radius of emulsion droplets with oil concentrations from 0 to 20%, and it can be seen that the curves are almost the same. In FIG. 10B the retraction rate (the radial retraction velocity normalized by the maximum expansion diameter) is measured for various cases: hexadecane emulsions, silicone oil emulsions and water droplets impacting LIS surfaces. It can be seen that the retraction rate, in these experiments, does not depend on the Weber number. It is also observed that the presence of oil in the emulsion or as a lubricating layer only slightly decreases the retraction rate (less than 20% in all cases). In these experiments, the retraction rate does not depend on the vertical suction force and is set by the inertial-capillary balance. The impregnated oil only minimally affects the retraction phase in these experiments, introducing a suction force that arises when the droplet starts moving vertically and getting detached from the surface.

To estimate the ridge size, the length h of the oil layer spreading on the droplet is estimated by balancing the driving capillary force with viscous dissipation in the growing oil layer

$${σ}_{o} \sim {μ}_{o} \frac{{V}_{o}}{e} h \sim {μ}_{o} \frac{{h}^{2}}{e {τ}_{c}}$$

where V₀ is the velocity of the oil spreading, e is the thickness of the oil layer and σ_{c} is the contact time (Inset of FIG. 9B). Here it is assumed that assume e=t. The size of the ridge is then

$$h \sim {( \frac{{σ}_{o} t {τ}_{c}}{{μ}_{o}} )}^{1 / 2}$$

The suction force as the droplet as the droplet starts moving vertically after the retraction is approximated as

$${F}_{s} \sim \frac{{σ}_{o}}{h} {R}_{0}^{2} ϕ$$

where φ is the coverage of the surface by oil (FIG. 7D) and

$${τ}_{c} \sim 2.6 {( \frac{ρ {R}_{0}^{2}}{σ} )}^{1 / 2}$$

is the contact time.

The scaling law below naturally follows:

$${F}_{s} \sim {( \frac{{σ}_{o} {μ}_{o}}{t {τ}_{c}} )}^{1 / 2} {R}_{0}^{2} ϕ$$

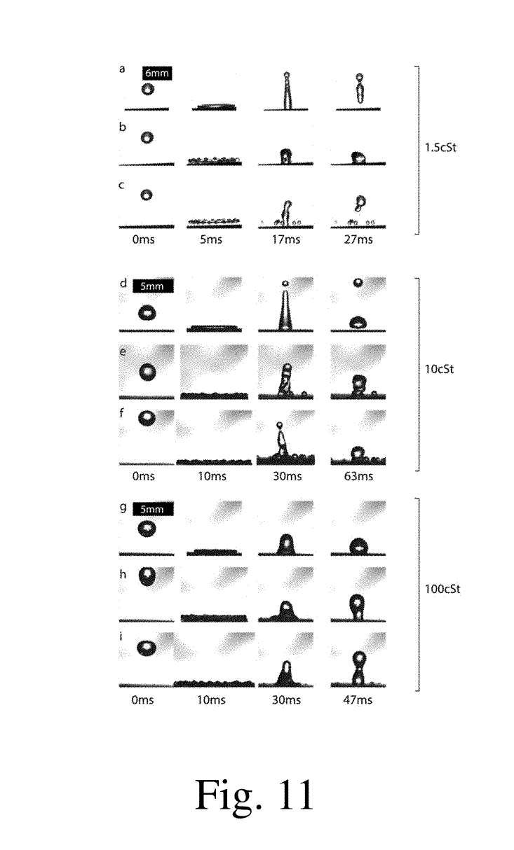

Fₛ is independent of the impact speed. FIG. 9B shows graphs of the suction force for emulsion droplets of a constant size (2 mm) and for oil concentrations of 5 and 20%. Another curve shows the bouncing force, with a sharp drop around We=30, which may correspond to the drop in the restitution coefficient. One explanation of the origin of the bouncing-sticking-bouncing transition becomes apparent: in the case of a 20% emulsion, the oil suction force may overcome the bouncing force after the sharp drop in the restitution coefficient and cause droplets to stick. The kinetic energy keeps growing and for We>50, the bouncing force may overcome the suction force again. The experimental data show that the droplets studied here stick in the 30<We<50 window and bounce otherwise. For lower oil concentrations (5% here), the suction force may remain lower than the bouncing force for many Weber numbers, resulting in few cases where droplets stick. For a concentration of 10%, some droplets are observed to stick but the window is narrower than for a 20% concentration. The above formulas for kinetic energy, bouncing force and suction force are not meant to be exact predictions but rather trends to explain the observed behavior. Given the variability in the transition of the restitution coefficient as well as in the coverage value, some experimental outliers are still observed, but it is believed that the model may capture the general trend. Similar behavior and bouncing-sticking-bouncing transitions are also observed for water droplet impacts on liquid-impregnated surfaces (LIS), which are dip-coated beforehand and have a complete layer of oil inside their texture (FIG. 11). The LIS case represents a particular case of the previous model, where the coverage φ=1. The ridge formation and the suction force may still occur, and therefore, the previous model may be appropriate for predicting sticking or bouncing for the case of liquid-infused surfaces as well.

### Dynamics of Impregnation of the Surface by Emulsion Drops

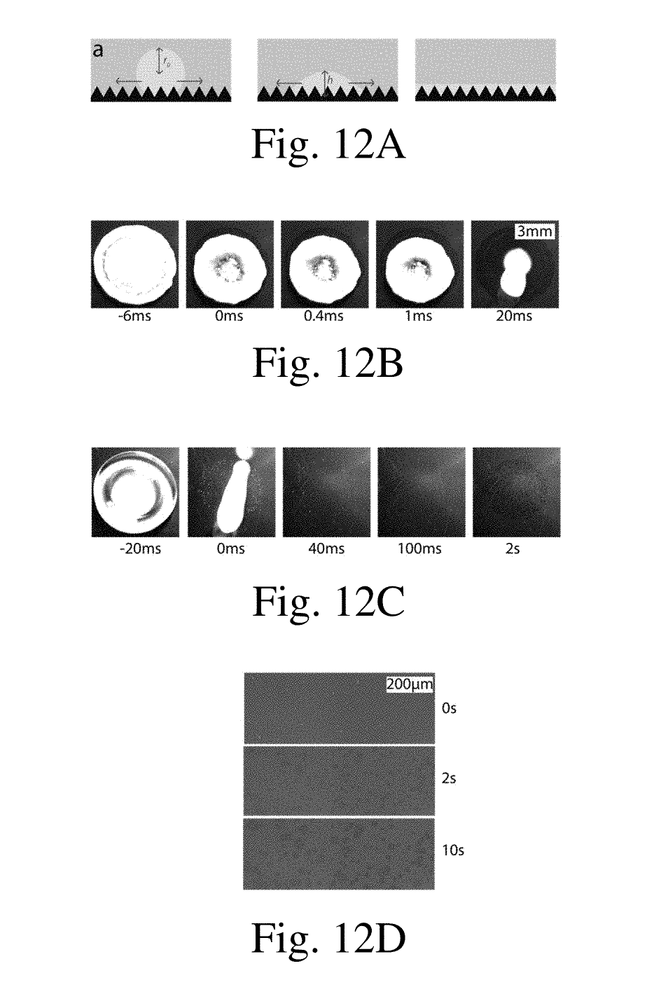

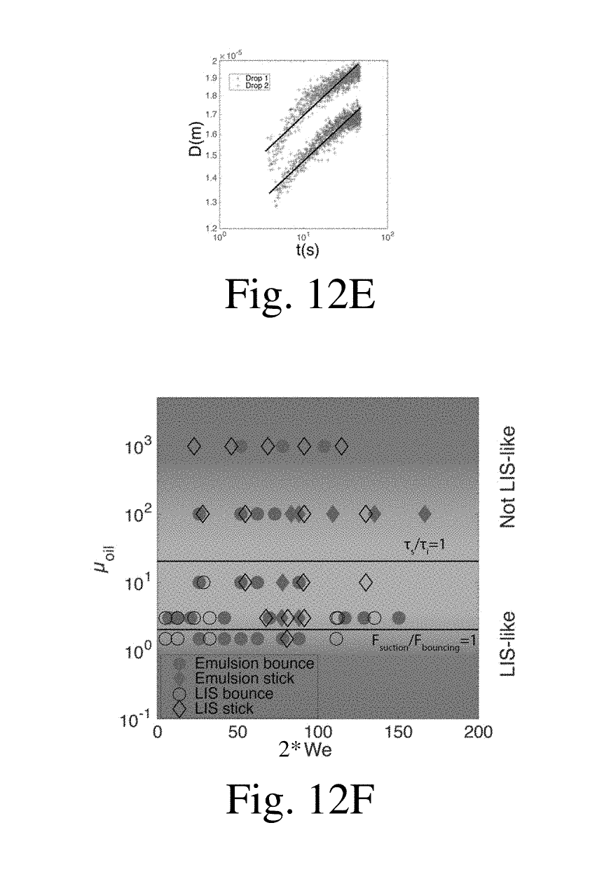

In the case of hexadecane, a relatively low-viscosity oil, it was assumed that the impregnation of the surface by the oil droplets was immediate. However, for more viscous oils, the kinetics of the impregnation may become limiting. As oil droplets touch the surface during the contact time of the droplet, they may start spreading into the texture (FIG. 12A). This spreading takes a certain time; here, its dynamics are modeled using the Navier-Stokes equation in the oil droplet.

$$\frac{dP}{dx} \sim {μ}_{o} \frac{{d}^{2} v}{{dy}^{2}}$$

$$\frac{σ h}{{r}^{3}} \sim {μ}_{o} \frac{v}{{h}^{2}} -> v \sim \frac{σ {h}^{3}}{{μ}_{o} {r}^{3}}$$

### So

$$r ( t ) \sim {( {Ω}^{3} \frac{σ}{{μ}_{o}} t )}^{1 / 10}$$

with

Ω=4/3πr₀³˜hr²

The time at which h=t (roughness height) is

$${τ}_{s} = \frac{{Ω}^{2} {μ}_{o}}{{h}^{5} σ} = \frac{{μ}_{o}}{σ} {( \frac{4}{3} π )}^{2} \frac{{r}_{0}^{6}}{{h}^{5}}$$

For an oil droplet initial radius of 700 nm and a height of 150 nm, this time is about 27 ms (contact time) when the viscosity is 20 cSt. Beyond that viscosity, the state of the surface when the emulsion droplet is retracting may start deviating from a partially liquid-infused surface. In some cases, the droplets may not have sufficient time to impregnate the surface. In those cases, oil droplets may act as solid obstacles on the surface and/or may not generate a suction force that enhances retention of impacting droplets. Top view snapshots of emulsion impacts with various viscosities are shown in FIGS. 12B-12D. For μ₀=10 cSt, as the droplet starts retracting, the oil has already spread into the texture. However, for μ₀=1000 cSt, individual spherical droplets can be seen after the droplet retracts and bounce, and they take a longer time to spread. In the intermediate regime of μ₀=100 cSt, only a few individual spherical oil drops can be observed as the droplet retracts.

FIG. 12D shows snapshots of lower speed imaging of an impact of a 1000 cSt emulsion under a microscope. Individual oil droplets with a spherical shape can be seen at first; later, these oil droplets can be observed spreading into the surface. The oil droplets were at the limit of the microscope resolution so the smallest ones could not be seen. The evolution of the contact diameter of some of the larger droplets was tracked; the graph for two of these droplets is shown in FIG. 12E. In these experiments, the diameter grows as t^{1/10} and the pre-factor in the expression is 1.2 10⁻⁵ ms^{-1/10}, which is close to the theoretical pre-factor given by the model (10⁻⁵ ms^{-1/10}).

FIG. 12F shows experimental outcomes of emulsion impacts at different viscosities (with a concentration of 10%), as well as the impacts of water droplets on oil-infused surfaces with oils of the same viscosities.

As predicted, for low viscosities, the observed suction force is low and cannot overcome the bouncing force due to the droplet's inertia. In that regime, it is possible that all droplets may bounce off. All of the emulsion with μ₀=1.5 cSt bounced, and water droplets impacting an LIS with the same oil only stick in a very narrow window of We (possibly due to their higher coverage factor φ). As the viscosity increased, bouncing-sticking-bouncing transitions were observed. For higher viscosities, when τₛ>τ_{contact}, the behavior diverged between emulsions and LIS. In the case of emulsions, it is believed that the oil droplets did not have time to impregnate the surface and did not form an oil layer capable of generating a suction force after the retraction. For μ₀=1000 cSt, emulsion droplets bounced for all Weber numbers. It is believed that this may be because there may not be oil in the surface texture. Therefore, for LIS surfaces where the oil layer is already present, and at higher viscosities, the suction force may be capable of overcoming the bouncing force for some, many, or all Weber numbers. It was observed that all water droplets stuck when impacted on surfaces infused with 100 cSt and higher viscosity silicone oil. At high oil viscosities in LIS, viscous dissipation during the retraction may become non-negligible and also contribute in arresting droplets.

There are certain beneficial ranges of viscosities where droplets stick, possibly because the generated suction force is strong enough to arrest droplets and/or because the impregnation of oil from emulsions is fast enough to occur during the contact time of the droplet impact. There are also certain beneficial ranges of Weber number, as discussed above and in FIGS. 9A-9B, possibly because, at these Weber numbers, viscous forces may be able to overcome the inertia of the droplet and prevent it from bouncing. Sprays formulated such as the velocity, size of the droplets and viscosity of the oil makes them in the optimal Weber number and/or viscosity regimes may be beneficial.

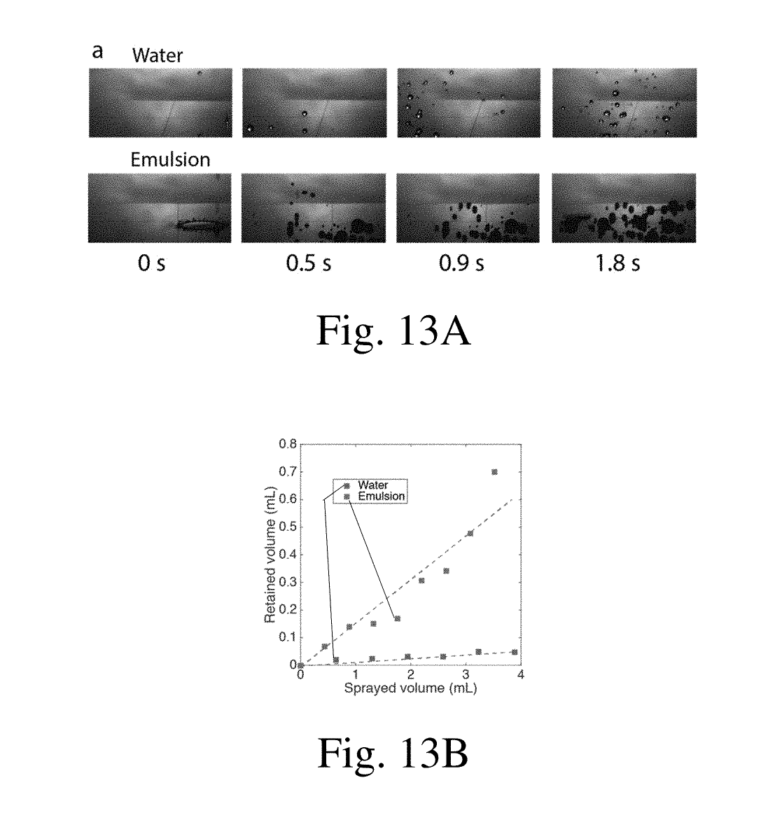

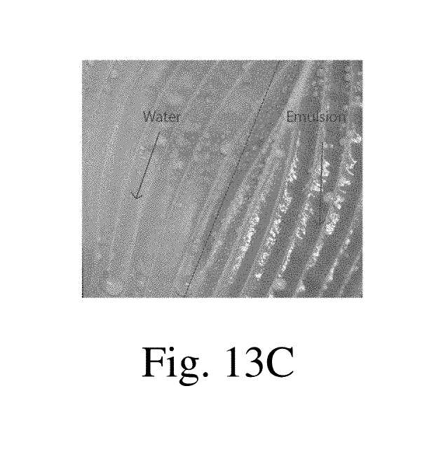

Finally, macroscopic experiments with sprays were performed. FIG. 13A shows snapshots of high-speed videos of water and emulsion (20% hexadecane) sprays impacting a superhydrophobic surface. As predicted, all water droplets bounce off and the surface remains dry. However, many emulsion droplets stick to the surface, and an accumulation of liquid on the surface is observed. Sparse sprays were used in these experiments for better visualization. To quantitatively capture the efficiency of emulsion sprays, the retained volume of liquid on the surface for a fixed amount of sprayed liquid was measured. The retention was determined by weighing the surface after each spray. The results are shown in FIG. 13B. The retained volume was observed to continuously increase for both water and emulsions but the rate of retention was observed to be 10 times higher in the case of emulsions. FIG. 13C qualitatively shows similar results on a hydrophobic hosta leaf. The right side of the leaf was sprayed with an emulsion and was completely covered with a uniform layer of liquid. The left side was sprayed with water and remained mostly dry, with only a few small droplets sticking to the surface.

### Conclusion

In this Example, a new method of sticking droplets to superhydrophobic surfaces has been explored. It has been found that, for a certain range of oil viscosity, impinging emulsion droplets can create partly liquid-impregnated surfaces in-situ, when emulsified oil droplets penetrate the surface texture. It is possible that this oil layer leads to additional viscous dissipation during the impact. For a certain interval of Weber numbers, viscous dissipation may overcome kinetic energy and prevents droplet rebound. It is believed that this happens at the onset of splashing, possibly due to the sharp drop in restitution coefficient as splashing starts to occur. It is believed that this technique is efficient in spray retention. This method can be used in agriculture for emulsified oil-based pesticides. By selecting advantageous values for oil concentration, droplet size and impact velocity, retention of sprays could be largely improved to eliminate runoff and environmental pollution by pesticides.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article comprising:
oil disposed on a surface comprising one or more protrusions, wherein:
the oil is deposited from an emulsion,
the emulsion comprises droplets comprising the oil dispersed within a fluid comprising water, and
a ratio of an average radius of the droplets comprising the oil to an average height of the protrusions is from 0.01 to 100.

2. An article comprising:
oil disposed on a surface comprising one or more protrusions, wherein:
the oil is deposited from droplets comprising an emulsion,
the droplets comprising the emulsion have an average radius R and an average density ρ;
the emulsion comprises droplets comprising a fluid comprising the oil dispersed within a fluid comprising water;
the droplets comprising the oil have an average radius r₀;
the fluid comprising the oil has a viscosity μ₀, a surface tension σ₀, and an average concentration in the emulsion C₀;
the fluid comprising water has a surface tension σ_{w};
the surface protrusions have an average height h;
the droplets comprising the emulsion have an average velocity v when the droplets comprising the emulsion contact the surface;
$$\frac{16 π}{26 {σ}_{o}} * {( \frac{{σ}_{w}}{ρ {R}^{3}} )}^{\frac{1}{2}} * \frac{{r}_{o}^{6}}{{h}^{5}} < 10 ; and$$
$$\frac{20 {C}_{o}^{\frac{2}{3}}}{3 {h}^{\frac{3}{2}}} * {( \frac{{σ}_{o}^{2} {σ}_{w} {μ}_{o}^{2}}{{ρ}^{5}} )}^{\frac{1}{4}} * \frac{1}{{R}^{\frac{3}{4}} {v}^{2}} > 0.3 .$$

3. A method of depositing oil on at least a portion of a surface, comprising:
contacting the entirety of the portion of the surface with an emulsion such that, after contact with the emulsion, an area fraction of the surface is covered by oil,
wherein the area fraction of the surface covered by oil after contact with the emulsion is within 50% of an area fraction desired to be covered by oil, and
wherein the area fraction desired to be covered by oil is from 0 to 1.

4-5. (canceled)

6. An article as in claim 2, wherein an average Weber number of the droplets comprising the emulsion is from 50-70.

7. An article as in claim 1, wherein the emulsion further comprises an agricultural chemical.

8. An article as in claim 7, wherein the agricultural chemical is a pesticide.

9. An article as in claim 1, wherein the surface comprises a portion of a plant.

10. An article as in claim 9, wherein the portion of the plant is a leaf.

11. An article as in claim 1, wherein the surface has a roughness of from 1 nm to 500 microns.

12. An article as in claim 1, wherein the surface has a roughness of from 20 nm to 50 microns.

13. An article as in claim 1, wherein a contact angle of water with the surface, prior to contact with the emulsion, is from 90° to 180°.

14. An article as in claim 1, wherein contact of the surface with the emulsion causes from 30% to 100% of the surface exposed to the emulsion to be covered by the emulsion.

15. An article as in claim 1, wherein contact of the surface with the emulsion causes the surface to hold from 0.5 mL/cm² to 100 mL/cm² of the emulsion.

16. An article as in claim 1, wherein contact of the surface with the emulsion causes the surface to hold from 4 mL/cm² to 40 mL/cm² of the emulsion.

17. An article as in claim 1, wherein the surface is contacted with a composition after being contacted with the emulsion.

18. An article as in claim 17, wherein the composition comprises water.

19. (canceled)

20. An article as in claim 2, wherein
$$\frac{16 π}{27 {σ}_{o}} * {( \frac{{σ}_{w}}{ρ {R}^{3}} )}^{\frac{1}{2}} * \frac{{r}_{o}^{6}}{{h}^{5}} < 2.$$

21. An article as in claim 2, wherein
$$\frac{20 {C}_{o}^{\frac{2}{3}}}{3 {h}^{\frac{3}{2}}} * {( \frac{{σ}_{o}^{2} {σ}_{w} {μ}_{o}^{2}}{{ρ}^{5}} )}^{\frac{1}{4}} * \frac{1}{{R}^{\frac{3}{4}} {v}^{2}} > 2.$$

22. An article as in claim 2, wherein contact of the surface with the droplets causes at least a portion of the droplets to be retained on the surface.

23. (canceled)

24. A method as in claim 3, wherein the area fraction of the portion of the surface covered by oil is within 20% of the area fraction desired to be covered by oil.

25-75. (canceled)
